(19) United States
(12) Patent Application Publication
(10) Pub. No.: US 2016/0101139 A1
(43) Pub. Date: Apr. 14, 2016

(54) TOPICAL TREATMENT FOR USE AS A SLEEP AID

(71) Applicant: Lou Paradise, Rhinebeck, NY (US)

(72) Inventor: Lou Paradise, Rhinebeck, NY (US)

(21) Appl. No.: 14/881,348

(22) Filed: Oct. 13, 2015

Related U.S. Application Data
(60) Provisional application No. 62/063,923, filed on Oct. 14, 2014.

(51) Int. Cl. A61K 36/28; A61K 36/77; A61K 36/81; A61M 21/02; A61K 31/198; A61K 36/75; A61K 35/583; A61K 36/185; A61K 36/22; A61K 45/06

(52) U.S. Cl. CPC A61K 36/28; A61K 36/22; A61K 36/77; A61K 36/81; A61K 45/06; A61K 31/198; A61K 36/75; A61K 35/583; A61K 36/185; A61M 21/02

(57) ABSTRACT

The present invention relates to a homeopathic sleep aid composition that may be applied topically. A composition may contain a synergistic blend of snake venom and relaxation-inducing herbs. A composition may facilitate relaxation by acting like an antihistamine, a GABA modulator, or an aromatherapeutic compound. A homeopathic composition may be applied topically on any part of the skin and even on open wounds. A homeopathic composition may also be formulated in an oral composition or intranasal composition. A homeopathic composition may aid people who have or do not have pain to fall asleep and stay asleep.

### CROSS REFERENCE TO RELATED APPLICATION

This application claims priority to the U.S. Provisional Patent Application Ser. No. 62/063,923, filed on Oct. 14, 2014 and entitled Topical Treatment for Use as a Sleep Aid, the contents of which are relied upon and incorporated herein by reference.

### FIELD OF INVENTION

The present invention relates to methods and apparatuses for a topical homeopathic composition sleep aid. More particularly, the present invention relates to the topical application of a homeopathic composition to the skin to induce relaxation and help people fall asleep and stay asleep.

### BACKGROUND

There are three common classes of sleep aids: benzodiazepines, histamine antagonists, and homeopathic herbal remedies.

Benzodiazepines such as Lorazepram (Ativan) increase the frequency for opening of chloride ion channels on GABA receptors to enhance the effects of the GABA neurotransmitter. This allosteric modulation of GABA receptors causes an increase in GABA, which produces relaxing effects. Benzodiazepines are effective sleep aids, but a patient may develop resistance and dependency.

Histamine antagonists such as diphenhydramine hydrochloride (Benadryl) cross the blood brain barrier then blocks histamine from binding to histamine receptors in the central nervous system and results in drowsiness. Histamine antagonists may cause grogginess and likely do not provide for a full night's sleep.

Homeopathic herbal remedies induce relaxation and have sedative effects due to the following. Chamomile contains apigenin, which increases GABA by binding to GABA receptors. Valerian contains GABA analogs. Passionflower contains beta-carboline harmala alkaloid MAO inhibitors. Hops contains methylbutanol and dimethyl vinyl carbinol. Ingestion of herbs can upset the stomach and cause indigestion, making a person more uncomfortable and possibly making it more difficult to relax and fall asleep.

Homeopathic sleep aids can be taken orally or be topically applied to the skin. Topically applied herbs such as the aforementioned can be infused in a carrier oil, balm, lotion, gel, or cream and rubbed on the skin. These topically applied sleep aids often contain aromatic compounds that help induce relaxation. These sleep aids may also contain DMSO along with herbs to facilitate pinocytosis, enhance medicine absorption, and decrease pain and inflammation. However, there are downsides to using homeopathic herbal remedies. Aromatic compounds can irritate the nasopharynx with sub-chronic use. Not an effective concentration of medicinal compounds will pass into the blood stream. The herbal remedy may not also last through the whole night.

The following are examples of topically applied homeopathic sleep aids. Natural Patches of Vermont relaxing sleep formula essential oil body patches contain lavender, chamomile, and jasmine. The patches are placed on the body and activated by body heat. Warrior sleep topical sleep aid contains DMSO, olive oil, distilled water, MSM, magnesium oil, lavender oil, lecithin, roman chamomile oil, coconut oil, and melatonin. Deep Sleep aromatherapy oil or cream contains bergamot, chamomile Roman, marjoram sweet, neroli, valerian root, and petitgrain. Neurobiologix calming cream contains water (Reverse Osmosis), Caprylic/Capric Triglycerides, Emulsifying Wax NF, Magnesium (Citrate), Glycerin, Butyrospermum Parkii (Shea Butter), Cetearyl Alcohol (and) Ceteareth-20, L-Tyrosine, Dimethicone, L-Theanine, Glyceryl Stearate, 5-HTP, GABA, Phenoxyethanol, Disodium EDTA, Xanthan Gum, Lavandula Angustifolia (Lavender).

The following are examples of orally ingested homeopathic sleep aids. King Bio Sleep aid (mouth spray) contains Aconitum napellus, Arsenicum album, Avena sativa, Belladonna, Camphora, Cinchona officinalis, Coffea cruda, Cypripedium pubescens, Hyoscyamus niger, Passiflora incarnata, Pulsatilla, Valeriana officinalis. Each ingredient is in equal volumes of 10×, 30×, and 100× potencies in a bio-energetically enhanced pure water base. Bach rescue sleep contains Helianthemum nummularium, Clematis vitalba, Impatiens glandulifera, Prunus cerasifera, Omithogalum umbellatum, Aesculus hippocastanum all in a vegetable carrier oil with natural flavoring. Boiron Quietude contains Hyoscyamus niger, Nux moschata, Passiflora incarnata, and Stramonium.

The safety, efficacy, and longevity of some sleep remedies are unknown. Additionally, many of the herbal concoctions would likely require formulation by a highly trained Chinese medicine practitioner. Research has shown that herbal sleep aids are more effective when they contain several herbs that act synergistically rather than individually. A Chinese study recommended a concoction of stir-fried spine date seed, Szechwan lovage rhizome, and prepared thinleaf milkwort root; white peony root, golden thread, and stir-fried spine date seed; and Asiatic cornelian cherry fruit with fresh rehmannia DOI:10.1007/s11655-011-0841-9.

An example of a homeopathic sleep aid has a pharmaceutical composition of tryptophan, L-tryptophan, lavender, chamomile, valerian root, passionflower, lemon balm, inositol, magnesium, Humulus lupus, hops extract, St. John's wort, or melatonin.

Another example is an herbal treatment for insomnia and forgetfulness that includes lily, lotus, mangosteen, mushrooms, and licorice. A traditional Chinese medicine prescription cream that contains ginseng, poria, and honey that is heated and made into a thick paste has also been used as a sleep aid.

What is needed therefore is a long-lasting natural sleep aid that is non-habit forming, does not have an irritating odor, and will help people fall asleep, stay asleep, and feel energized and regenerated in the morning.

### SUMMARY

Accordingly, the present invention provides a homeopathic composition sleep aid that may be applied topically, orally ingested or sprayed intranasally. The topical homeopathic composition may be more effective and last longer than other herbal sleep aids without becoming habit-forming. The topical homeopathic composition induces relaxation to allow for a full night's sleep.

One general aspect includes a method for aiding sleep, the method including the steps of identifying an effective application situs for sleep aid on human body and applying a topical composition onto the effective application situs for sleep aid on human body. The topical composition may include an effective amount of a combination of herbs or extracts of arnica montana, rhus toxicodendron and aesculus hippocastanum mixed with an effective amount of belladona, in a pharmaceutically acceptable carrier.

Implementations may include one or more of the following features. The method may include examples where the topical composition additionally includes a vasodilator component including one or more of: primrose oil, rosemary, ginger, hamamelis, horse chestnuts, yohimbe, and niacin 1-ornithate. The method may include examples where the topical composition includes between about 0.5 to 5% by weight of the vasodilator component. The method may include examples where the topical composition additionally includes one or both of snake venom and snake venom extract. The method may additionally include the step of applying a treatment bandage including a layer of the composition over the effective application situs for sleep aid on human body.

The method where the topical composition additionally includes one or more mobilizers of white blood cell activity including one or more of: lachesis ninta venom, crotalus horridus venom, naja venom, red chinese ginseng, siberian ginseng and korean ginseng, goldenseal, chamomile, and barberry. The method may also include examples where the topical composition includes between about 0.5 to 5% by weight of the one or more mobilizers of white blood cell activity.

One general aspect includes a sleep aid bandage for facilitating sleep. The sleep aid bandage may include a sealing layer formable to a contour of an effective application situs for sleep aid, a substrate fixedly attached to the sealing layer, and a treating composition on a first surface of the substrate fixedly attached to the sealing layer. The treating composition may include an effective amount of a combination of herbs or extracts of arnica montana, rhus toxicodendron and aesculus hippocastanum mixed with an effective amount of belladona, in a pharmaceutically acceptable carrier.

The details of one or more embodiments of the invention are set forth in the accompanying drawings and the description below. The accompanying drawings that are incorporated in and constitute a part of this specification, illustrate several embodiments of the invention and, together with the description, serve to explain the principles of the invention: Other features, objects, and advantages of the invention will be apparent from the description, drawings and the claims herein.

### DESCRIPTION OF THE DRAWINGS

The foregoing and other features and advantages of the invention will be apparent from the following, more particular description of preferred embodiments of the invention, as illustrated in the accompanying drawings.

FIG. 1 illustrates exemplary methods of applying topical application of topical homeopathic composition on the skin to induce sleep.

FIG. 2 illustrates exemplary embodiments of topical application of topical homeopathic composition before bedtime.

FIG. 3 illustrates exemplary embodiments of intranasal use of homeopathic sleep aid to induce sleep.

### DETAILED DESCRIPTION

The present invention relates generally to a homeopathic composition sleep aid. In particular, the present invention provides a topical application, such as a cream or gel that may be applied on or into the skin, wherein the topical application comprises one or more a vasodilator, stimulator of lymphatic activity, or mobilizers. Preferred embodiments include ingredients derived from natural sources, and in some aspects, natural sources processed through homeopathic steps. Homeopathic steps may include, for example, filtration and dilution.

More specifically, the components of the composition may comprise a vasodilator, such as, for example, Arnica Montana, primrose oil, rosemary, ginger, Hamamelis, horse chestnuts, Yohimbe, niacin L-ornithate, and the like; a stimulator of lymphatic activity, such as, for example, Echinacea Augustofolia, St. John's Wort, Belladonna, Devils Claw, Yellow Dock, Burdock, North American Ginseng, Wild Indigo, Pleurisy Root, and Pokeweed; and mobilizers of white blood cell activity, such as, for example, Lachesis Ninta venom, Crotalus horridus venom, Naja venom, Red Chinese Ginseng, Siberian Ginseng and Korean Ginseng, Goldenseal, Chamomile, and Barberry. Each of the components of the composition relating to vasodilation, lymphatic activity and white blood cell activity may be used in the amount of between about 0.5 to 5% by weight, and preferably, between about 0.5 to 2.0% by weight.

In some embodiments, the homeopathic composition may comprise Caprylyl Glycol, which may function as a humectant and/or stabilizer. Caprylyl glycol may be plant derive or synthetic. Caprylyl glycol may increase antimicrobial activity of other preservatives, and may be used in conjunction with phenoxyethanol and chloroxylenol. The preservative blend may work on the skin's surface to give it a soft and smooth texture.

In some aspects, the homeopathic composition may comprise Caprylic/Capric Triglyceride, which may be produced by chemical reactions between various fatty acids and glycerol (synthetic glycerine). This largely synthetic mixed triester may be derived from coconut oil and glycerin and may comprise an emollient, dispersing agent, and solvent. In some aspects, Caprylic/Capric Triglyceride may comprise spreading properties that may facilitate gliding of the homeopathic composition on the skin.

In some embodiments, the homeopathic composition may comprise Caprylhydroxamic Acid, which may take place of traditional preservatives. Caprylhydroxamic Acid has the best length (C8) of C chain to promote degradation of cell membrane structures. Caprylhydroxamic Acid may maintain a unionized state in acid to neutral and may perform at a neutral pH. Caprylhydroxamic Acid may have excellent efficacy of fungistatic agents.

In some aspects, the homeopathic composition may comprise Citrus Aurantium Amara (Bitter Orange) Flower Water may comprise flavonoids, which are anti-inflammatory, antibacterial, and antifungal. Orange flower water contains hydrophilic properties and may act as a natural surfactant and emulsifier to enhance the application of skin lotions and other cosmetics. In some aspects, the orange flower water may be safe to use directly on the skin, without requiring dilution, and may be used on blotchy, irritated skin. Orange flower water is a by-product of distillation and may be used in perfumery, which may provide a soothing aroma that may be enhance the relaxing effects of the homeopathic composition. In some aspects, orange flower water may be used medicinally to reduce heart rate and palpitations, which may encourage sleep and calm the digestive tract. Orange flower water is smoothing, soothing, and brightening due to the skin regenerative and vitamin-rich properties of organic citrus flowers.

In some embodiments, the homeopathic composition may comprise Lauric Acid. Lauric Acid is a main fatty acid in coconut oil, which may be used as a moisturizer for the skin and may reduce redness and flaking of skin with minimal adverse side effects.

In some aspects, the homeopathic composition may comprise Citrus Aurantium dulcis (orange) peel oil, which may be extracted from the peels of both sweet and sour oranges by cold compression. Orange oil extract may have inflammatory qualities, which may detoxify congested skin and soothe dry or irritated skin, such as may occur in shingles, dermatitis, and acne outbreaks. Orange oil extract may increase the production of collagen and may reduce fine lines and puffiness, which may be effective as a concurring or post acupuncture treatment, particularly around acupuncture points.

In some embodiments, the homeopathic composition may comprise Coenzyme A, which may be adapted from pantothenic acid and adenosine triphosphate. Biologically speaking, this coenzyme plays a vital role in the synthesis and oxidation of fatty acids. Coenzyme A may function as a skin conditioner, emollient, and solvent, and in some aspects, coenzyme A may stimulate collagen production.

In some aspects, the homeopathic composition may comprise Dehydroacetic Acid. As a mild acid, dehydroacetic acid may work with benzyl alcohol as a preservative and antimicrobial to provide a broad protection from contamination. Dehydroacetic acid may function as a fungicide and/or bactericide, which, in some embodiments, may be used in personal care products at a maximum concentration of 0.6%.

In some embodiments, the homeopathic composition may comprise Ginkgo Biloba Leaf Extract, which may be a potent antioxidant that may improve blood flow.

In some aspects, the homeopathic composition may comprise Gluconolactone, which is an ester of gluconic acid and is composed of multiple water-attracting hydroxyl groups. Gluconolactone may hydrate skin and enhance the degree of moisturization of the homeopathic composition. Gluconolactone is a polyhydroxy acid (PHA) that is capable of chelating metals and may also function by scavenging free radicals. In some embodiments, as a gentle ingredient, gluconolactone may be directly applied on sensitive skin.

In some embodiments, the homeopathic composition may comprise Hydrolyzed Lupine Protein, which is a hydrolyzed protein from the seeds of the lupine plant (lupinus). The seeds are high in proteins and contain 35-45% of all the essential amino acids. The hydrolyzed lupine protein may have a restructuring and regenerating effect on skin. Lupine peptides are a blend of penta- and hexa-peptides derived from lupin flower protein that may function as an MMP (matrix metalloprotease) inhibitor, which may prevent MMP activity following exposure of the skin to sunlight. Accordingly, the lupine peptides blend may prevent collagen and elastin (the main proteins making up connective tissue) breakdown catalyzed by MMP activity, which may facilitate renewal of the skin's outer layer, which may stimulate the production of collagen & elastin. In some aspects, the hydrolyzed lupine protein may reduce scarring that may be caused by lesions associated with shingles.

In some aspects, the homeopathic composition may comprise Hydroxypropyl tetrahydropyrantriol, which is a sugar-protein hybrid made from xylose, a sugar found abundantly in beech trees. As a series of amino acids, Hydroxypropyl tetrahydropyrantriol is small enough to penetrate the skin, which may allow for deeper and more effective penetration of the homeopathic composition. Hydroxypropyl tetrahydropyrantriol stimulates the production of glycosaminoglycan's (GAGs), or mucopolysaccharides. GAGs, which are an important component of connective tissue and may increase production on an extracellular matrix.

Snake venom and venom extracts have been found to have pro- and anti-inflammatory activity to dull pain, increase circulation, and reduce edema. Snake venom and venom extracts have been found to stimulate the production of both pro- and anti-inflammatory cytokines. The pro-inflammatory cytokines are produced which may lead to an increase in a localized heat sensation. Anti-inflammatory cytokines are produced which may help in eliminating pain and edema. Any discomfort associated with pain from an inflammatory response may be dulled. Any dulling of pain from snake venom may result in relaxation and may aid a person to fall asleep.

Lachesis muta snake venom may produce a hemorrhaging effect that may increase blood flow and circulation in the area where a topical homeopathic sleep aid may be applied. Crotalus horridus snake venom may produce anti hemorrhagic effects that may decrease inflammation and blood flow in an area where a topical homeopathic sleep aid may be applied. Snake venom from species of Naja may induce edema and a tingly sensation that may dull pain in an area where a topical homeopathic sleep aid may be applied. Different snake venom combinations and in various dilutions may work synergistically to reduce pain and induce sleep.

For example, a topical homeopathic sleep aid containing snake venom may be applied to a wound site or any other part of the body. In some embodiments, a topical homeopathic sleep aid with snake venom may make a wound site more inflamed for a few minutes. Another effect of a topical homeopathic sleep aid with snake venom may be where a wound site that may be irritated and inflamed may become less painful and less swollen and red. A wound may be on or below the skin. In some aspects, a topical homeopathic composition may modulate GABA receptors and produce a sleep-aid mechanism similar to popular prescription sleep aids. In some aspects, a topical homeopathic composition may block histamine responses to promote relaxation and sleep.

Furthermore, a topical homeopathic composition may also contain essentials oils such as tea tree oil that have natural antibacterial properties. A topical homeopathic composition may also contain aspirin, which may aid in pain reduction and increased blood flow at the body's pressure points. Essential oils and traditional analgesics such as aspirin may enhance vasodilation and pain relief. Furthermore, essential oils and analgesics may synergistically enhance the effects of a homeopathic composition as well as prevent any dermal infections that may occur from needle insertions or wound exposure. As a result, a homeopathic composition containing essential oils and/or aspirin may relieve pain and inflammation, which may induce relaxation and may allow a person to experience a deep sleep. In some aspects, a topical homeopathic composition may help a person fall asleep, stay asleep for at least 8 hours, and wake up without any groggy feelings.

In some aspects, a topical homeopathic composition may reduce risk of irritation to the skin or nasopharynx while also promoting instantaneous relaxation from intranasal application. In intranasal formulation may be in a gel form or liquid form. An intranasal formulation may be sprayed in one or both nostrils and may or may not have aromatherapy effects. An intranasal formulation may be sprayed as early as 20 minutes before going to bed to right before going to bed. An intranasal composition may also be in an ointment form and may be applied just inside the nostrils and on and around the nose. An intranasal formulation may release soothing botanicals that may reduce inflammation, pain, and sinus pressure which may help a person relax, fall asleep, and stay asleep.

In some aspects, a topical homeopathic composition may include extended release beads containing the active ingredients in a topically-applied cream, wherein a single application may last an extended period. In some such examples, extended-release beads may reduce additional steps, reapplications, or products that may be used in conjunction with the topical homeopathic composition application. Some active ingredients suspended in a lotion, cream, gel, or salve may take effect instantaneously and last for 4 hours when applied topically on the skin.

A homeopathic composition containing extended release beads may be applied on a wound, at chakra and pressure points, or on any part of the body. At 4 hours, some extended release beads may liquefy from body heat and release more active ingredients that may produce sleep-aid and pain relief effects for another 4 hours. Extended release beads may eliminate a need for an application of a bandage or patch. Extended release beads may be microscopic or macroscopic. Extended release beads may have tenacious properties that may help extended release beads adhere to the skin, so extended release beads may not rub off throughout the night.

In some aspects, an orally-ingested formulation of the homeopathic sleep aid may contain a capsule containing finely ground herbs or a compacted tablet containing homeopathic dilutions of some ingredients. A formulation may also be in the form of a liquid such as a tonic, tincture, or tea. A homeopathic composition may be taken orally 5 minutes before going to bed and may last for up to 8 hours. The oral route, as well as the intranasal route, of exposure to some active ingredients may allow some ingredients to pass through the blood brain barrier to facilitate the sleep-aid and relaxation mechanism of some ingredients. An orally ingested or intranasal form of a homeopathic sleep aid may induce sleep in a mechanism similar to an antihistamine mechanism or GABA modulator. An orally ingested or intranasal homeopathic sleep aid may or may not contain ingredients that need to pass the blood-brain barrier in order to induce relaxation.

In some aspects, a topical homeopathic composition may further comprise ingredients useful in aromatherapy, which may enhance the sleep aid effects of the homeopathic composition. A homeopathic composition may include essential oils or extracts of lavender, vanilla, and chamomile that may not cause irritation. The addition of fragrance may induce relaxation and sleep more quickly and efficiently than if a topical homeopathic sleep aid is applied on the skin alone. A homeopathic composition used in an intranasal route of exposure may also contain aromatherapeutic compounds such as natural essential oils or synthetic aromatic compounds. In some embodiments, a lozenge form of a homeopathic sleep aid homeopathic composition may also contain aromatherapeutic compounds such as menthol that may induce relaxation and deep sleep.

In some aspects, a topical homeopathic sleep aid may induce relaxation systemically due to muscle tension release and opening of the body's energy channels. In some examples, a topical homeopathic sleep aid may comprise ingredients capable of promoting penetration and absorption of any active ingredients into the body. A topical homeopathic composition may comprise ingredients which may include large molecules that are not readily absorbed into the skin but may need to be absorbed into the bloodstream to produce a relaxing and sleep-aid effect. Therefore, a component of a carrier surfactant, emollient, or gel may facilitate pinocytosis, so a large-molecule ingredient may penetrate through the skin and may be absorbed into the bloodstream if required for that particular ingredient's mechanism of action. As a result, vasodilation and opening of energy channels may occur. Vasodilation and opening of the body's energy channels may induce relaxation by relieving muscle tension and spasms.

For example, a person with restless leg syndrome may apply a topical homeopathic sleep aid to the skin where the quadriceps and gastrocnemius are located. A topical homeopathic sleep aid may penetrate the skin and may be absorbed into the dermis where blood vessels may be located. Blood vessels may dilate and may increase blood flow and energy flow to the quadriceps and gastrocnemius which may result in released tension and spasms. A topical homeopathic sleep aid may physically relax a person and may allow a person to lie still and fall asleep. In some aspects, a topical homeopathic sleep aid may mentally relax a person and may allow a person to lie still and fall asleep. In some embodiments, a topical homeopathic sleep aid that may release muscle tension may allow a person to sleep through a whole night and may allow a person to awaken without any feelings of grogginess.

In some aspects, a topical homeopathic sleep aid may be applied in regions of the body where penetration and/or absorption may be more efficient, such as parts of the body with larger surface area like the chest or back. In some aspects, particularly in areas of the body where penetration may be difficult, such as the knees or elbows, an additive or a preparation step may increase penetration. For example, an area may be prepared by a simple alcohol swab which may remove surface contaminants that may inhibit penetration.

Another example may be exfoliation before applying a topical homeopathic composition. In some aspects, a topical homeopathic composition may also include particles that may contain exfoliate properties. Exfoliates may be physical particles that may be botanical such as ground seeds or synthetic such as microbeads. Exfoliates may also be molecular such as glycolic acid. In such aspects, an exfoliate in a topical homeopathic sleep aid may remove skin from the stratum corneum which may improve penetration of ingredients into the dermis and may improve absorption of ingredients into the bloodstream. An example of a use is when a homeopathic sleep aid with an exfoliate may be applied to the upper back where a person may be experiencing muscle tension and rubbed in for several minutes. A homeopathic composition with an exfoliate may be wiped off while still may be producing pain relief and circulatory effects to locally and/or systemically induce relaxation and sleep.

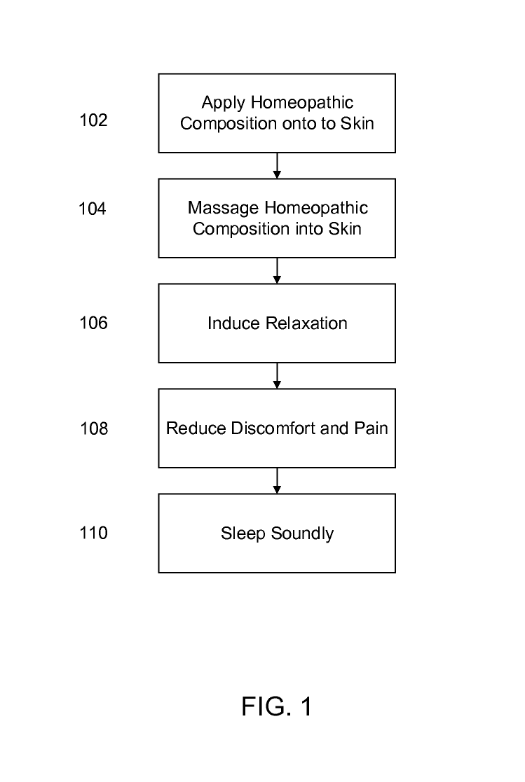

Referring to FIG. 1, an exemplary methods of the topical application of a homeopathic sleep aid is illustrated. In some methods, a person may apply a homeopathic sleep aid to the skin 102. A person may massage a homeopathic sleep aid on the skin 104. In some methods, relaxation may be induced 106. In some methods, pain and discomfort may be reduced 108. In some methods, a person may sleep soundly 110. In some aspects, a person may apply a predefined amount of topical homeopathic composition on a part of the body such as the chest. A topical homeopathic composition may be rubbed in, simply spread over the skin, or layered. An application may occur within a predefined period prior to intended sleep, such as 30 minutes before going to bed. A homeopathic composition may produce sleep-aid effects for up to 8 hours.

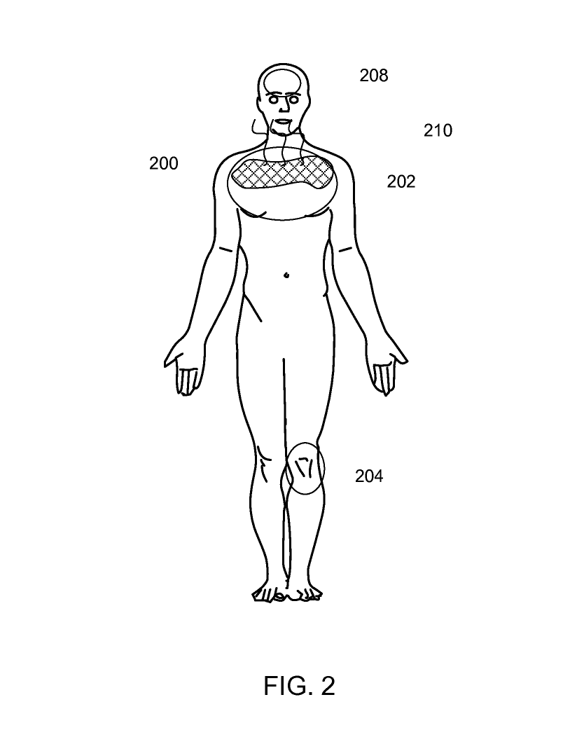

Referring to FIG. 2, an exemplary embodiment of some locations of application of a homeopathic sleep aid are illustrated. In some embodiments, a homeopathic sleep aid 202 may be applied to the chest or other area of the body with a large surface area 200. A homeopathic sleep aid may produce aromatherapy vapors 210. A homeopathic sleep aid may calm a person's mind and nerves 208. A homeopathic sleep aid as in 202 may be applied to an area of pain and inflammation 204. In such application to an area of pain and inflammation 204, a homeopathic sleep aid may increase blood flow, which may induce relaxation and may induce sleep.

In some aspects, a topical composition may be used as a sleep aid in conjunction with pain management. In some aspects, a source of the pain may comprise damage from a fall or a lesion-causing disease, such as shingles, wherein pain may be reduced or at least unaffected by a direct application of the topical composition to a lesion. In some such examples, a direct application to an open wound may allow for a more effective penetration of one or both the pain relieving and/or sleep facilitating ingredients as well as quicker relief.

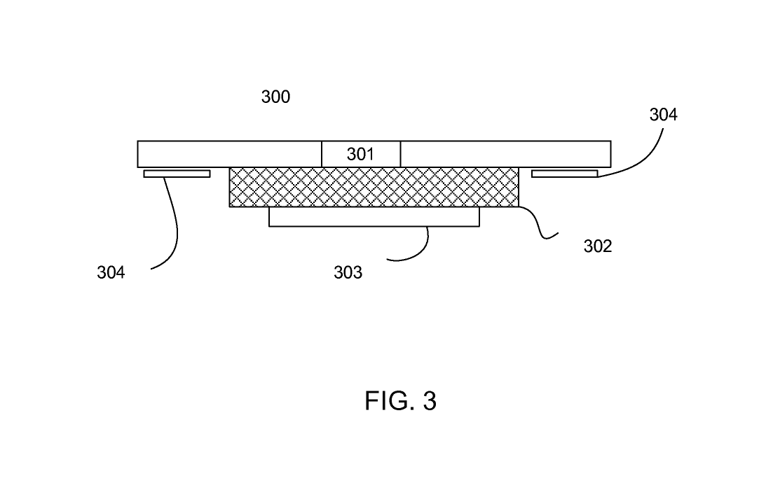

Referring now to FIG. 3, in some embodiments, a treatment bandage 300 may include a substrate 302 dosed with the homeopathic composition 303. The substrate 302 may be comprised of a porous portion impregnated with homeopathic composition 303. The substrate 302 may therefore include one or both of a natural fiber, such as a cotton or other plant based fiber and a synthetic fiber or gel. The substrate 302 may also include a dissolvable starch that may dissolves into the skin. The substrate 302 may be fixedly attached to a sealing layer 301 that generally provides a boundary between the homeopathic composition 303 and an ambient environment. The sealing layer 301 may include a polymer, vinyl, latex, plastic or other material formable to a contour of a human body part.

In some embodiments, the sealing layer may include an adhesive layer 304 for removably attaching the substrate to a body part in a fashion that allows the homeopathic composition 303 to be brought into contact with the skin of a patient and maintained in contact with the skin. The sealing layer allows that homeopathic composition 303 to remain relatively undisturbed by an ambient environment.

In some embodiments, a person may use a nasal spray cartridge that may be inserted halfway into the nostrils. A person may squeeze or press down on an applicator to release a homeopathic sleep aid, wherein the homeopathic sleep aid may travel down a person's nasopharynx. A homeopathic sleep aid may pass the blood brain barrier. In such embodiments, a nasal homeopathic sleep aid may induce sleep due to an antihistamine, GABA, or aromatherapy mechanism.

In some embodiments, a person may ingest orally a pill, capsule, or lozenge. In some embodiments, a pill may travel down the nasopharynx and into the gastrointestinal tract, wherein one or more ingredients of a homeopathic sleep aid may be absorbed into the bloodstream. In some embodiments, a person may drink a tonic or apply a sublingual tincture or lozenge of a homeopathic sleep aid which may induce sleep.

### ILLUSTRATIVE EXAMPLES

Illustrative examples of homeopathic composition formulas are listed and described below. These are exemplary only and should not be considered as limiting.

### Example 1

### A Homeopathic Gel Prepared by Admixing the Following Ingredients

| Ingredient | Wt. % |
|---|---|
| Carbomer 940 | 2.10 |
| Xantham gum | 0.15 |
| Propylene glycol | 51.94 |
| Dipropylene glycol | 10.00 |
| Ethoxydiglycol | 15.00 |
| Dimethylisosorbide | 10.00 |
| Aloe Vera gel | 2.00 |
| Surfactant | 0.05 |
| Amica Montana | 2.50 |
| Belladona | 2.50 |
| Rhus toxicodendron | 2.00 |
| Aescuius hippocastanum | 1.76 |

Although the specific activity of each of either plants or herbs have been recognized, it has been surprisingly found that the combination as now claimed has been found to produce the desired effect. The composition is applied to the limb having pain 1-8 times per day.

### Example 2

### A Cream was Formed by Admixing the Following Ingredients

| Ingredient | |
|---|---|
| Aesculus hippocastanum extract | 6 mcg/g |
| Amica Montana extract | 6 mcg/g |
| L-arginine | 6 mcg/g |
| Echinacea augustfolia extract | 6 mcg/g |
| Rhus toxicondendron extract | 6 mcg/g |
| Ruta graveoleus extract | 6 mcg/g |
| Graphites | 6 mcg/g |
| Crotalus horridus extract | 0.08 mcg/g |
| Heloderma horridum extract | 0.08 mcg/g |
| Lachesis extract | 0.08 mcg/g |
| Naja extract | 0.08 mcg/g |
| Isopropyl myristate | 30 mg/g |
| Brij 72 | 2 mg/g |
| Brij 72IS | 2 mg/g |
| Water | 83 mg/g |
| Methyl paraben | 0.2 mg/g |
| Propyl paraben | 20 mcg/g |
| Glycerine | 3 mg/g |
| 20% NaOH | 3 mg/g |
| Dowcil 200 | 50 mcg/g |
| Akodel 112 | 2 mg/g |

The cream may be used to treat cracked skin on feet or anhidrosis, which is associated with CRPS.

### Example 3

### A Lotion is Prepared by Admixing the Following Ingredients

| Ingredient | Wt. % |
|---|---|
| Ginger | 1.10 |
| Propylene Glycol Stearate | 6.50 |
| Isocetyl alcohol | 5.00 |
| PEG-100 Stearate | 1.20 |
| Water | 69.90 |
| Echinacea augustfolia extract | 3.00 |
| Methyl paraben | 0.20 |
| Propylene glycol | 12.00 |
| Sorbitan palmitate | 0.60 |
| Arnica Montana | 3.00 |
| Aesculus hippocastanum extract | 2.00 |
| Barberry | 1.00 |
| Mate extract | 0.50 |

If desired, 3% by weight capsaicin can be added. The lotion can be used to treat a patient suffering from fibromyalgia.

### Example 4

### Preparation of a Gel

| Ingredient | Wt. % |
|---|---|
| Arnica Montana | 5.00 |
| Primrose oil | 3.00 |
| Arginine base (10% solution) | 5.00 |
| (Ajinomoto) | |
| Carbopol 940 | 0.40 |
| Butylene glycol | 6.50 |
| Echinacea augustfolia | 3.00 |
| Chamomile glycolic extract | 3.00 |
| Crotalus horridus venom | 0.50 |
| Preservative | 0.10 |
| Fragrance | 0.10 |
| Deionized water | g.s. |

To 20 ml of water with stirring is added the Carbopol 940. The mixture is stirred until hydration is complete and then butylene glycol is added. The arginine base is then added to the mixture. The remaining ingredients are mixed together and added to the first mixture. The mixing is continued until uniform. The composition can be applied 1-8 times daily to reduce the pain resulting from CRPS.

### Example 5

### A Gel is Prepared by Admixing the Following Ingredients

| Ingredient | Wt. % |
|---|---|
| Propylene Glycol | 43 |
| Polyacrylic acid | 2.1 |
| Dipropylene Glycol | 16 |
| Xantbam Gum | 0.15 |
| Etboxydiglycol | 15 |
| Dimetbylisosorbide | 10 |
| Ascorbic Acid | 2 |
| Chloroxylenol | 0.2 |
| Linoleamidopropyl PG- | 1.5 |
| diammonium chloride phosphate | |
| Glyceretb 4.5 Lactate | 2 |
| Naja venom extract | 2 |
| Echinacea augustfolia | 2 |
| Octoxynol-9 | 0.5 |
| Primrose Oil | 2 |
| Cocamidopropyl PG-dimon | 1 |
| chloride phosphate | |
| Water | 6 |
| Ginger | 0.44 |

Ingredients 1 and 2 are mixed to disperse and form a gel. About 80% of ingredient 3 is mixed with ingredient 4, added to the gel slightly heated with admixture. The balance of 3 is mixed with ingredients 5-17 and added to the gel at 38 degrees. After mixing, the pH is adjusted to about 4 and then the gel is brought to room temperature. Arnica Montana may also be added to have a plurality of dual acting ingredients. Capsaicin together with the ginger can be used to relieve pain.

### CONCLUSION

A number of embodiments of the present invention have been described. While this specification contains many specific implementation details, there should not be construed as limitations on the scope of any inventions or of what may be claimed, but rather as descriptions of features specific to particular embodiments of the present invention.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

While the invention has been described in conjunction with specific embodiments, it is evident that many alternatives, modifications and variations will be apparent to those skilled in the art in light of the foregoing description. Accordingly, this description is intended to embrace all such alternatives, modifications, and variations as fall within its spirit and scope.

Although shown and described in what is believed to be the most practical and preferred embodiments, it may be apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims.

What is claimed is:

1. A method for aiding sleep, the method comprising the steps of:
identifying an effective application situs for sleep aid on human body; and
applying a topical composition onto the effective application situs for sleep aid on human body, wherein the topical composition comprises an effective amount of a combination of herbs or extracts of Arnica Montana, Rhus toxicodendron and Aesculus hippocastanum mixed with an effective amount of belladona, in a pharmaceutically acceptable carrier.

2. The method of claim 1 wherein the topical composition additionally comprises a vasodilator component comprising one or more of: primrose oil, rosemary, ginger, Hamamelis, horse chestnuts, Yohimbe, and niacin L-ornithate.

3. The method of claim 2 wherein the topical composition comprises between about 0.5 to 5% by weight of the vasodilator component.

4. The method of claim 2 wherein the topical composition additionally comprises a component comprising a stimulator of lymphatic activity comprising one or more of: Echinacea Augustofolia, St. John's Wort, Belladonna, Devils Claw, Yellow Dock, Burdock, North American Ginseng, Wild Indigo, Pleurisy Root, and Pokeweed.

5. The method of claim 2 wherein the topical composition comprises between about 0.5 to 5% by weight of the component comprising a stimulator of lymphatic activity.

6. The method of claim 4 wherein the topical composition additionally comprises one or more mobilizers of white blood cell activity comprising one or more of: mobilizers of white blood cell activity, including one or more of: Lachesis Ninta venom, Crotalus horridus venom, Naja venom, Red Chinese Ginseng, Siberian Ginseng and Korean Ginseng, Goldenseal, Chamomile, and Barberry.

7. The method of claim 6 wherein the topical composition comprises between about 0.5 to 5% by weight of the one or more mobilizers of white blood cell activity.

8. The method of claim 2 wherein the topical composition additionally comprises a humectant comprising Caprylyl glycol in conjunction with phenoxyethanol and chloroxylenol.

9. The method of claim 8 wherein the topical composition comprises between about 0.5 to 5% by weight of the humectant.

10. The method of claim 2 wherein the topical composition additionally comprises a preservative comprising Caprylhydroxamic Acid.

11. The method of claim 2 wherein the topical composition additionally comprises one or more flavonoids, which are anti-inflammatory, antibacterial, and antifungal.

12. The method of claim 2 wherein the topical composition additionally comprises one or both of snake venom and snake venom extract.

13. The method claim 12 wherein the snake venom comprises a Lachesis muta snake venom.

14. The method claim 12 wherein the snake venom comprises a Crotalus horridus snake venom.

15. The method of claim 12 wherein the composition comprises a gel.

16. The method of claim 12 wherein the composition comprises a lotion.

17. The method of claim 12 wherein the composition comprises a cream.

18. The method of claim 1 additionally comprising the step of applying a treatment bandage comprising a layer of the composition over the effective application situs for sleep aid on human body.

19. A sleep aid bandage for facilitating sleep, the sleep aid bandage comprising:
a sealing layer formable to a contour of an effective application situs for sleep aid;
a substrate fixedly attached to the sealing layer; and
a treating composition on a first surface of the substrate fixedly attached to the sealing layer, the treating composition comprising an effective amount of a combination of herbs or extracts of Arnica Montana, Rhus toxicodendron and Aesculus hippocastanum mixed with an effective amount of belladona, in a pharmaceutically acceptable carrier.

20. The sleep aid bandage of claim 19 wherein the composition comprises a gel.
